(19) United States
(12) Patent Application Publication
(10) Pub. No.: US 2009/0112242 A1
(43) Pub. Date: Apr. 30, 2009

(54) EAR CURETTE

(75) Inventor: Yu-Yueh KAO, Taipei (TW)

Correspondence Address: PATENTTM.US, P. O. BOX 82788, PORTLAND, OR, 97282-0788, US

(73) Assignee: HIGHPLUS INTERNATIONAL CO., LTD., Taipei (TW)

(21) Appl. No.: 12/177,087

(22) Filed: Jul. 21, 2008

(30) Foreign Application Priority Data
Oct. 24, 2007 (TW) 096217790

(51) Int. Cl. A61F 11/00

(52) U.S. Cl. 606/162

(57) ABSTRACT

An ear curette has a shaft and a cleaning portion. The shaft has a distal end. The cleaning portion has a disk and a bar. The bar has a distal end and a proximal end. The proximal end of the bar is narrower than, is formed on and protrudes from distal end of the shaft. The disk is formed on the distal end of the bar. When in use, the disk of the cleaning portion can be deposited in the ear canal first and then pulled out of the ear canal to scrape out earwax and is easily cleanable.

### BACKGROUND OF THE INVENTION

1. Field of the Invention

The present invention relates to an ear curette, particularly to clean earwax from ear canals.

2. Description of the Prior Arts

With reference to FIG. 5, a conventional ear curette is used to clear wax build up from an ear canal and consists of a shaft (12) and a ring assembly (10). The shaft (12) has a distal end and a proximal end. The ring assembly (10) is mounted around the distal end of the shaft (12) and may be a rigid material and may be further coated with a resilient material such as rubber or the like. The ring assembly (10) consists of multiple parallel rings (11). The parallel rings (11) are mounted around the distal end of the shaft (12). Each two of the rings (11) define a gap for accommodating earwax so as to be scraped out from the ear canal by withdrawing the shaft (12). However, only one ring (11) being most proximal to the distal end, of the shaft (12) effectively scrapes earwax from the ear canal, and the other rings (11) possess little or no effect on cleaning the earwax since they have a same diameter and merely follow a same path. Furthermore, the other rings (11) might cause sanitary problems because the gap between each two rings (11) is too narrow to be cleaned effectively, resulting in earwax accumulation between the gaps between each two rings (11).

With further reference to FIG. 6, another ring assembly (20) is a resilient material. The ring assembly (20) has a hollow body and multiple rings (21). The body of the ring assembly (20) is mounted around the distal end of the shaft (12). The rings (21) are elastic and formed concentrically around the body of the ring assembly (20). Each two of the rings (21) define a gap for accommodating earwax. Nevertheless, the ring assembly (20) still has the sanitary problem due to similar cleaning difficulties.

To overcome the shortcomings, the present invention provides an ear curette to mitigate or obviate the aforementioned problems.

### SUMMARY OF THE INVENTION

The main objective of the invention is to provide an ear curette that is easy to clean.

An ear curette has a shaft and a cleaning portion. The shaft has a distal end. The cleaning portion has a disk and a bar. The bar has a distal end and a proximal end. The proximal end of the bar is narrower than, is formed on and protrudes from distal end of the shaft. The disk is formed on the distal end of the bar. When in use, the disk of the cleaning portion can be deposited in the ear canal first and then pulled out of the ear canal to scrape out earwax and is easily cleanable.

Other objectives, advantages and novel features of the invention will become more apparent from the following detailed description when taken in conjunction with the accompanying drawings.

### BRIEF DESCRIPTION OF THE DRAWINGS

FIG. 1 is a side view of an ear curette in accordance with the present invention;

FIG. 2 is a perspective view of the ear curette in FIG. 1;

FIG. 3 is an operational side view of the ear curette in FIG. 1, showing the ear curette cleaning an ear canal;

FIG. 4 is a perspective view of an ear curette in accordance with the present invention, showing an ear curette being accommodated in a corresponding cover in accordance with the present invention;

FIG. 5 is a side view of an ear curette in accordance with the prior art; and

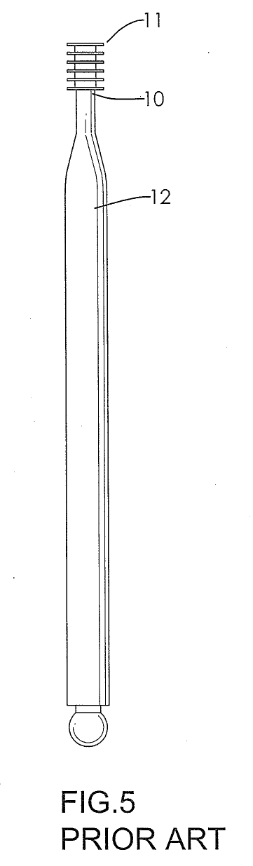

FIG. 6 is a side view in partial section of another ear curette in accordance with the prior art.

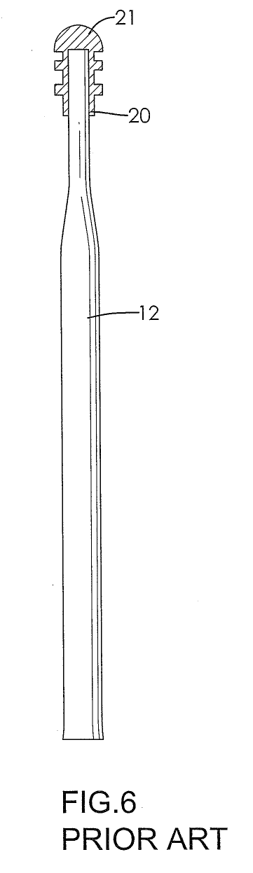

### DETAILED DESCRIPTION OF THE PREFERRED EMBODIMENTS

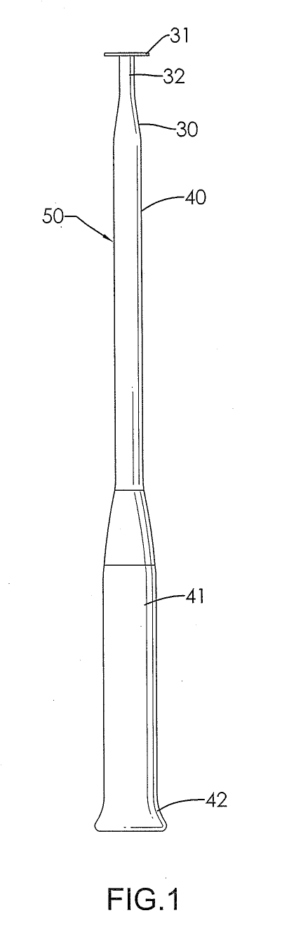

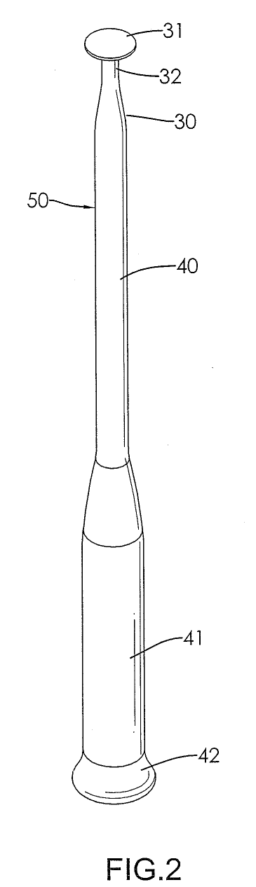

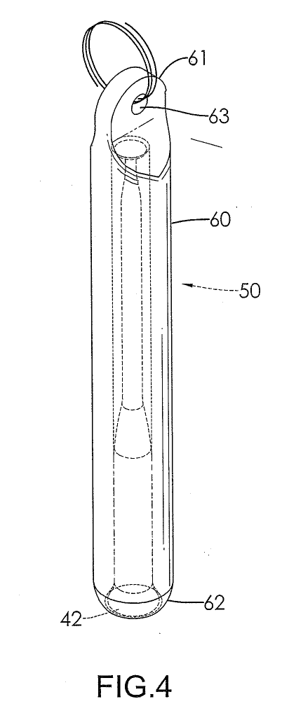

With reference to FIGS. 1, 2 and 4, an ear curette (50) in accordance with the present invention has a shaft (40) and a cleaning portion (30) and may be implemented with an optional cover (60).

The shaft (40) has a proximal end, a distal end, an optional handle (41) and an optional butt (42). The handle (41) may be cylindrical and has a distal end and a proximal end. The distal end of the handle (41) is formed on the proximal end of the shaft (40). The butt (42) is formed at the proximal end of the handle (41) and is larger than the handle (41) to aid handling.

The cleaning portion (30) has a bar (32) and a disk (31). The bar (32) is narrower than, is formed on and protrudes from the distal end of the shaft (40) and has a distal end. The disk (31) is circular and has an attaching surface and an outer surface. The attaching surface is attached to the distal end of the bar (32). The outer surface is opposite to the attaching surface and may be flat. When implemented, earwax is stored between the disk (31) and the bar (32) and is easily removed.

The cover (60) is a hollow cylinder and has an attaching end, an insertion end, an inner surface and a lobe (61). The inner surface is formed in the cover (60), defines a chamber for accommodating the shaft (40) and the cleaning portion (30) and has an optional holding segment (62). The chamber corresponds to and accommodates the ear curette (50) in accordance with the present invention. The holding segment (62) corresponds to the concave surface of the butt (42). The lobe (61) is formed on the attachment end of the cover (60) and has an annular edge and a through hole (63). The annular edge protrudes from the attachment end of the cover (60). The through hole (63) is formed centrally through the lobe (61) for an attachment device such as a key ring, chain or string to be passed through.

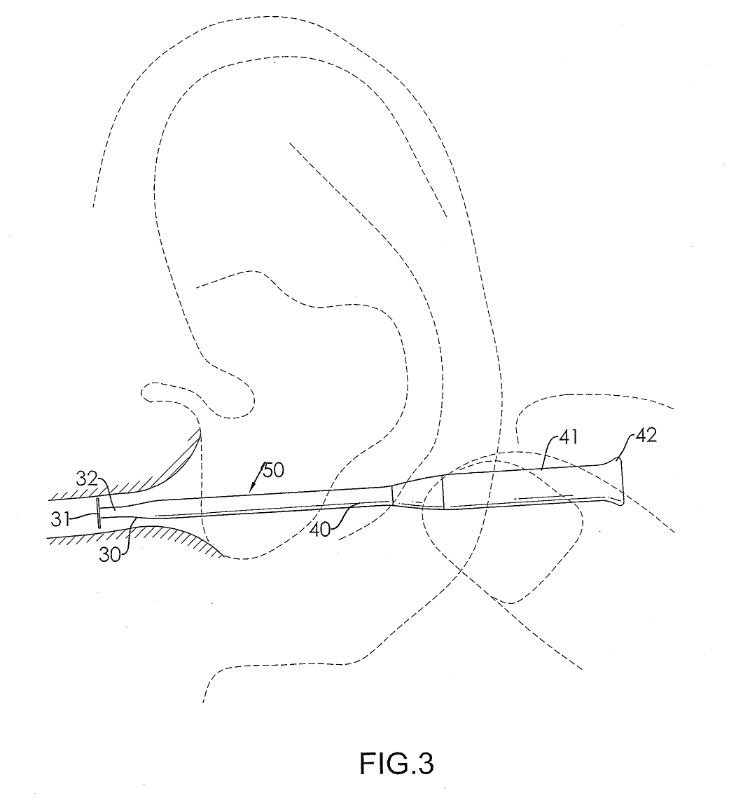

With further reference to FIG. 3, the ear curette (50) in accordance with the present invention is held by the handle (41). The cleaning portion (30) is deposited in an ear canal. Then, the disk (31) of the cleaning portion (30) is pulled out of the ear canal, such that earwax can accumulate on the attaching surface of the disk (31) to be carried out of the ear canal.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An ear curette comprising
an ear curette has
a shaft having a distal end and a proximal end; and
a cleaning portion having
a bar being narrower than and being formed on the distal end of the shaft and having a distal end; and
a disk being circular and having an attaching surface being attached to the distal end of the bar.

2. The ear curette of claim 1, wherein the shaft further has a handle being a cylindrical and having
a distal end being formed on the proximal end of the shaft; and
a proximal end.

3. The ear curette of claim 2, wherein the handle further has
a butt being larger than and being formed around the proximal end of the handle.

4. The ear curette of claim 1 further comprising
a cover being a hollow cylinder and having
an attaching end;
an insertion end; and
an inner surface being formed in the cover, defining a chamber accommodating the shaft and the cleaning portion.

5. The ear curette of claim 4, wherein the shaft further has
a handle being cylindrical and having
a distal end being formed on the proximal end of the shaft; and
a proximal end.

6. The ear curette of claim 5, wherein the handle further has
a butt being larger than and being formed around the proximal end of the handle.

7. The ear curette of claim 4, wherein the cover further has
a lobe being formed on the attachment end of the cover and having
an annular edge protruding from the attachment end of the cover; and
a through hole being formed centrally through the lobe.

8. The ear curette of claim 5, wherein the cover further has
a lobe being formed on the attachment end of the cover and having
an annular edge protruding from the attachment end of the cover; and
a through hole being formed centrally through the lobe.

9. The ear curette of claim 6, wherein the cover further has
a lobe being formed on the attachment end of the cover and having
an annular edge protruding from the attachment end of the cover; and
a through hole being formed centrally through the lobe.
